(19) United States
(12) Patent Application Publication
(10) Pub. No.: US 2021/0353613 A1
(43) Pub. Date: Nov. 18, 2021

(54) TREATMENT OF NEUROLOGICAL DISEASE

(71) Applicants: Aclipse One Inc., Radnor, PA (US); The University of Sheffield, Sheffield (GB)

(72) Inventors: Ning Shan, Chandler, AZ (US); Pamela Jean Shaw, Derbyshire (GB); Claude Ogoe, Monrovia, CA (US); Laura Ferraiuolo, Sheffield (GB)

(21) Appl. No.: 17/286,799

(22) PCT Filed: Oct. 18, 2019
(86) PCT No.: PCT/US19/56998

§ 371 Date: Apr. 19, 2021

Related U.S. Application Data
(60) Provisional application No. 62/747,961, filed on Oct. 19, 2018.

(51) Int. Cl. A61K 31/473; A61P 25/28

(52) U.S. Cl. CPC A61K 31/473; A61P 25/28

(57) ABSTRACT

The invention is directed to (6aS)-6-methyl-5,6,6a,7-tetrahydro-4H- dibenzo[de,g]quinoline-10,11-diol for the treatment of diseases mediated by protein misfolding of Cu/Zn Superoxide Dismutase (SOD1) or mediated by astrocyte toxicity affecting motor neuron survival.

### CROSS-REFERENCE TO RELATED APPLICATION

The present application claims the benefit of U.S. Provisional Application No. 62/747,961, filed Oct. 19, 2018, the disclosure of which is hereby incorporated by reference in its entirety.

### FIELD OF THE INVENTION

The present invention relates to a therapeutic agent and methods for the treatment of diseases mediated by mechanisms associated with Cu/Zn Superoxide Dismutase (SOD1) protein misfolding, or astrocyte toxicity affecting motor neuron survival.

### BACKGROUND OF THE INVENTION

Cu/Zn Superoxide Dismutase 1 (SOD1), HGNC:7782 http://www.ncbi.nlm.nib.gov/gene/4780, UniProtKB - P00441 (SODC_HUMAN), is a 32kDa ubiquitously expressed enzyme found in cells, more specifically the cytosol, nucleus, mitochondria, and peroxisomes, which dismutes toxic superoxide anions into oxygen and peroxide.

Mutant SOD1 enzymes, and a dysfunctional Proteostasis Network (PN), due, for example, to environmental factors, gene mutations/mutant proteins, and aging, drive misfolding of SOD1 enzymes. Persistent misfolding of SOD1 enzymes inhibits the ability of SOD1 to dismute superoxide, thus increasing the build-up of superoxide in cells which leads to oxidative stress. Terminally misfolded and aggregated SOD1, which is not cleared by either the Ubiquitin Proteasome System and/or autophagy eventually sequester proteins that are critical to cellular processes, co-sequester chaperones that maintain the PN, perturb intracellular trafficking, and disrupt cell membrane integrity.

Therefore, abnormal misfolding, terminally misfolded, and aggregated SOD1 enhance oxidative stress which damages lipid membranes, proteins, and nucleic acids, and drive degeneration of cells, which eventually leads to cell death.

Mitochondrial diseases that result from mitochondrial dysfunction increase the formation of reactive oxygen species (ROS) that results in oxidative stress. Excessive production of ROS exacerbates misfolded SOD1 which attenuates the ability of SOD1 to dismute excessive superoxide. This eventually leads to dysfunctional mitochondrial processes, degeneration of mitochondria and mitochondrial death.

Mitochondrial diseases include: Leigh syndrome, Alpers-Huttenlocher syndrome, Childhood myocerebrohepatopathy spectrum, Ataxia neuropathy spectrum, Myoclonic epilepsy myopathy sensory ataxia, Sengers syndrome, MEGDEL syndrome (also known as 3-methylglutaconic aciduria with deafness, encephalopathy and Leigh-like syndrome), Pearson syndrome, Congenital lactic acidosis (CLA), Leber hereditary optic neuropathy (LHON), Kearns-Sayre syndrome (KSS), Mitochondrial myopathy, encephalopathy, lactic acidosis and stroke-like episodes (MELAS) syndrome, Myoclonic epilepsy with ragged red fibres (MERRF), Neurogenic muscle weakness, ataxia and retinitis pigmentosa (NARP), Chronic progressive external opthalmoplegia (CPEO), Mitochondrial neurogastro-intestinal encephalopathy (MNGIE) syndrome, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked Bulbo-Spinal Atrophy, presenile dementia, depressive disorder, temporal lobe epilepsy, Fragile X Syndrome, Machado-Joseph Disease, Hereditary Leber Optic Atrophy, cerebrovascular accident, subarachnoid hemorrhage, and schizophrenia. The pharmacological intervention in the SOD1 pathway is a promising avenue for therapeutic intervention in diseases involving SOD1 protein misfolding, accumulation of misfolded SOD1 protein, and SOD1 protein aggregation. Therapeutics that reduce SOD1 misfolding represents a novel therapeutic strategy that could slow, halt, or reverse the underlying disease process in diseases involving the SOD1 pathway.

Recently, it was found that neighboring glial cells contribute to motor neuron degeneration through a non-cell autonomous process. Healthy motor neurons develop features typical of amyotrophic lateral sclerosis (ALS) pathology (i.e., ubiquitinated inclusions), when they are surrounded by mutant SOD1-expressing non-neuronal cells in a chimeric SOD1 mouse model. When the mutant SOD1 pathology was eliminated from the microglia, disease progression slowed by 50%. Targeted expression of mutant SOD1 in astrocytes does not result in an ALS phenotype, while silencing of the mutant gene slows disease progression. Primary astrocytes expressing mutant SOD1 have toxic effects on the surrounding motor neurons, indicating that astrocytes are physically exerting this toxicity, or are incapable of effectively supporting the motor neurons. Of great relevance for the ALS patient population, the same toxic/non-supportive properties have been found in patients that do not carry any mutation and develop sporadic ALS. More than 90% of ALS cases worldwide are sporadic.

There have been several potential mechanisms of astrocyte toxicity discovered using the mutant SOD1 mouse model and, more recently, astrocytes derived from sporadic patients, where SOD1 has been detected in its misfolded form. The finding that conditioned medium from astrocytes induces motor neuron loss has led to the idea that astrocytes secrete toxic factors. Several studies have attempted to identify these secreted toxic factors. Meanwhile, other evidence suggested that astrocytes might exert toxicity through a lack of support instead. The activation of pro-apoptotic factors such as BCL2-associated X protein (BAX) in motor neurons cultured with ALS astrocytes, the uncontrolled release of reactive oxygen species from ALS astrocytes and insufficient ion homeostasis resulting in hyperexcitability are all potential factors released by astrocytes. Astrocytes fail to provide motor neurons with metabolic substrates such as lactate and insufficient protection from toxic insults such as synaptic glutamate and activation of the pro-NGF-p75 signaling pathway. There has also been aberrant behavior observed in multiple astrocyte pathways that cross-talk with motor neurons, suggesting that this toxicity is the result of both a loss of physiological function and a toxic gain of function.

As shown above, astrocytes contribute to a series of toxic mechanisms affecting neuronal function and survival. Therefore, efforts have been taken to reduce astrocyte toxicity and improve the survival of cells such as motor neurons in drug development, especially targeting at neurodegenerative diseases.

### SUMMARY OF THE INVENTION

(6a5)-6-methyl-5,6,6a,7-tetrahydro-4H-dibenzo[de,g]quinoline-10,11-diol, the enantiomer of currently approved (6aR)-6-methyl-5,6,6a,7-tetrahydro-4H-dibenzo[de,g]quinoline-10,11-diol, is a weak dopamine antagonist and does not exhibit the side effects associated with dopamine agonism after administration. (6aS)-6-methyl-5,6,6a,7-tetrahydro-4H-dibenzo[de,g]quinoline-10,11-diol, also known as S-(+)-10,11-dihydroxyaporphine, is depicted by the following chemical structure:

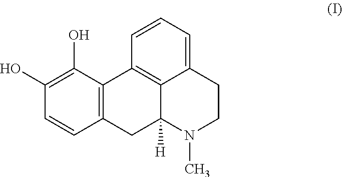 [SMILES: CN1CCc2cccc3c2[C@@H]1Cc1ccc(O)c(O)c1-3]

The present invention has experimentally demonstrated that (6aS)-6-methyl-5,6,6a,7-tetrahydro-4H-dibenzo[de,g]quinoline-10,11-diol can significantly reduce: SOD1 protein misfolding, accumulation of misfolded SOD1 protein, and SOD1 protein aggregation.

(6a5)-6-methyl-5,6,6a,7-tetrahydro-4H-dibenzo[de,g]quinoline-10,11-diol may be used in methods to reduce the frequency of SOD1 protein misfolding, to inhibit SOD1 protein misfolding, to refold misfolded SOD1, to reduce the accumulation of misfolded SOD1 protein, to reduce SOD1 protein aggregation, and to clear terminally misfolded and/or aggregated SOD1 in a cell. (6a5)-6-methyl-5,6,6a,7-tetrahydro-4H-dibenzo[de,g]quinoline-10,11-diol may further be used in methods for treating diseases mediated by SOD1 protein misfolding, accumulation of misfolded SOD1 protein, and SOD1 protein aggregation.

In one aspect, the present invention provides for a method of reducing the level of misfolded SOD1 in a cell, comprising a step of contacting the cell with an effective amount of (6a5)-6-methyl-5,6,6a,7-tetrahydro-4H-dibenzo[de,g]quinoline-10,11-diol.

In one aspect, the present invention provides for a method of reducing accumulation of misfolded SOD1 protein in a cell, comprising a step of contacting the cell with an effective amount of (6a5)-6-methyl-5,6,6a,7-tetrahydro-4H-dibenzo[de,g]quinoline-10,11-diol.

In one aspect, the present invention provides for a method of reducing SOD1 protein aggregation in a cell, comprising a step of contacting the cell with an effective amount of (6a5)-6-methyl-5,6,6a,7-tetrahydro-4H-dibenzo[de,g]quinoline-10,11-diol.

As used herein, the term “effective amount” means an amount that will result in the desired effect or result, e.g., an amount that will result in decreasing misfolded SOD1 levels, decreasing accumulation of misfolded SOD1, and/or decrease SOD1 protein aggregation.

In one embodiment, the method may be an in vitro method.

In another aspect, the present invention provides for a method of reducing the frequency of SOD1 protein misfolding, accumulation of SOD1 misfolded protein, or aggregation of SOD1 protein, and removal of terminally misfolded and/or aggregated SOD1 protein in a cell, comprising the step of contacting said cell with an effective amount of (6aS)-6-methyl-5,6,6a,7-tetrahydro-4H-dibenzo[de,g]quinoline-10,11-diol.

In one embodiment, the method may be an in vitro method.

In another aspect, the invention provides for a method of increasing cell lifespan, comprising the step of contacting said cell with an effective amount of (6aS)-6-methyl-5,6,6a,7-tetrahydro-4H-dibenzo[de,g]quinoline-10,11-diol.

In one embodiment, the method may be an in vitro method.

In one embodiment, the cell in one of the above aspects, or other aspect herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is in vitro, in vivo, or ex vivo.

In another embodiment, the cell is a diseased cell. In another embodiment, the cell is diseased cell from a patient suffering from a disease or disorder as defined below.

In another aspect, the invention provides for a method of treating an animal having a disease or disorder that would benefit from reducing the frequency of SOD1 protein misfolding, reducing the accumulation of SOD1 misfolded protein, or reducing aggregation of SOD1 protein, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides (6aS)-6-methyl-5,6,6a,7-tetrahydro-4H-dibenzo[de,g]quinoline-10,11-diol for use in the treatment of a disease or disorder by reducing the frequency of SOD1 protein misfolding, reducing the accumulation of SOD1 misfolded protein, or reducing aggregation of SOD1 protein.

In one embodiment, (6aS)-6-methyl-5,6,6a,7-tetrahydro-4H-dibenzo[de,g]quinoline-10,11-diol may be for use in the treatment of an animal having a disease or disorder characterized by increased frequency of SOD1 protein misfolding, increased accumulation of SOD1 misfolded protein, or increased aggregation of SOD1 protein.

In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising (6aS)-6-methyl-5,6,6a,7-tetrahydro-4H-dibenzo[de,g]quinoline-10,11-diol may be for administration to the animal in an effective amount.

In one embodiment, said animal is a mammal. In another embodiment, said mammal is a human or a non-human mammal. In a further embodiment, said mammal is a human.

In another embodiment, said disease or disorder is caused by protein misfolding, accumulation of misfolded proteins, or protein aggregation. In one embodiment, said disease or disorder is caused by SOD1 protein misfolding, accumulation of misfolded SOD1 protein, or SOD1 protein aggregation.

In another embodiment, the disease is a neurodegenerative disease. In another embodiment, said disease is selected from any one or more of: age-related macular degeneration, Alzheimer's disease (AD), amyotrophic lateral sclerosis (ALS), atherosclerosis, autism spectrum disorder (ASD), benign focal amyotrophy, cerebral infarction, Creutzfeldt-Jakob disease Crohn's disease, Duchenne's paralysis, Friedreich's ataxia, frontotemporal dementia (FTD), glaucoma, hereditary spastic paraplegia (HSP), Huntington's disease (HD), Inclusion Body Myopathy (IBM)inflammatory bowel disease, ischemia, Kugelberg-Welander syndrome, Lewy body diseases (LBD), Lou Gehrig's disease, multiple sclerosis (MS), myocardial infarction, necrotizing enterocolitis, Neurofibromatosis type I, Paget's disease of the bone (PDB), Parkinson disease (PD), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked Bulbo-Spinal Atrophy, presenile dementia, depressive disorder, temporal lobe epilepsy, Hereditary Leber Optic Atrophy, cerebrovascular accident, subarachnoid hemorrhage, and schizophrenia.

In one embodiment, the disease is amyotrophic lateral sclerosis (ALS).

In one embodiment, the disease is ALS caused by a mutation. In one embodiment, the disease is ALS caused by a mutation selected from: a C9orf72 mutation, a SOD1 mutation, or a sporadic mutation. In one embodiment, the disease is ALS caused by a SOD1 mutation.

In another aspect, the invention provides for a method of increasing lifespan or treating a disease or disorder resulting in accelerated aging or other abnormal aging process in an animal, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides for (6aS)-6-methyl-5,6,6a,7-tetrahydro-4H-dibenzo[de,g]quinoline-10,11-diol for use in the treatment of a disease or disorder resulting in accelerated aging or other abnormal aging process in an animal.

In another aspect, the invention provides for (6aS)-6-methyl-5,6,6a,7-tetrahydro-4H-dibenzo[de,g]quinoline-10,11-diol for use in the treatment of a disease or disorder by increasing lifespan of an animal.

In one embodiment, the disease or disorder is premature ageing.

In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising (6aS)-6-methyl-5,6,6a,7-tetrahydro-4H-dibenzo[de,g]quinoline-10,11-diol may be for administration to the animal in an effective amount.

In one embodiment, said animal is a mammal. In another embodiment, said mammal is a human or a non-human mammal.

In a related aspect, the invention provides for a method of treating premature aging due to chemical or radiation exposure in an animal, e.g., human, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In a related aspect, the invention provides for (6aS)-6-methyl-5,6,6a,7-tetrahydro-4H-dibenzo[de,g]quinoline-10,11-diol for use in the treatment of premature aging due to chemical or radiation exposure in an animal.

In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising (6aS)-6-methyl-5,6,6a,7-tetrahydro-4H-dibenzo[de,g]quinoline-10,11-diol may be for administration to the animal in an effective amount.

In one embodiment, the premature aging is due to exposure to chemotherapy, radiation therapy, or UV radiation. In a further embodiment, the UV radiation is artificial, e.g., tanning bed, or solar UV radiation, i.e., sun exposure. In one embodiment, the pharmaceutical composition is for topical administration on skin.

In another aspect, the invention provides for a method of improving the survival of cells by reducing the toxicity of astrocytes.

In one embodiment, the method may be an in vitro method.

In one embodiment, the cell is an animal cell, e.g., mammalian cell. In a further embodiment, said cell in a human cell or non-human cell. In a further embodiment, said cell is in vitro, in vivo, or ex vivo.

In a further embodiment, the astrocytes are associated with the cells. In one embodiment, the astrocytes are from the same source as the cells. In one embodiment, the astrocytes are from a patient suffering from a neurodegenerative disease. In one embodiment, the astrocytes are from a patient suffering ALS.

In another embodiment, the cell is a diseased cell. In another embodiment, the cell is diseased cell from a patient suffering from a neurodegenerative disease In another embodiment, the cell is a diseased cell from a patient suffering from ALS.

In another embodiment, the cell is a motor neuron cell. In another embodiment, the cell is motor neuron cell from a patient suffering from a neurodegenerative disease. In another embodiment, the cell is a diseased motor neuron cell from a patient suffering from a neurodegenerative disease. In another embodiment, the cell is a diseased motor neuron cell from a patient suffering from ALS. In another aspect, the invention provides a method of improving cell survival by reducing astrocyte toxicity in a cell, comprising a step of contacting the cell with an effective amount of an antioxidant compound

In another aspect, the invention provides a method of improving cell survival by reducing astrocyte toxicity in a cell, comprising a step of contacting the cell with an effective amount of (6aS)-6-methyl-5,6, 6a,7-tetrahydro-4H-dib enzo[de,g]quinoline-1 0, 1 1 -di ol .

In another aspect, the invention provides a method of treating an animal having a disease or disorder that would benefit from reducing astrocyte toxicity or improving cell survival, the method comprising the step of administering a therapeutically effective amount of a pharmaceutical composition comprising an antioxidant compound to said animal.

In another aspect, the invention provides a method of treating an animal having a disease or disorder that would benefit from reducing astrocyte toxicity or improving cell survival, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides an antioxidant compound for use in the treatment of a disease or disorder by reducing the toxicity of astrocytes and/or by increasing the survival of cells.

In one embodiment, the disease or disorder is a neurodegenerative disease or disorder, for example any of those listed hereinabove. In one embodiment, the disease or disorder is ALS. In one embodiment, the disease is ALS caused by a mutation selected from: a C9orf72 mutation, a SOD1 mutation, or a sporadic mutation.

In one embodiment, the cells are motor neuron cells.

In one embodiment, the astrocytes are associated with the cells.

In one embodiment, the antioxidant compound increases the survival of cells by reducing the toxicity of astrocytes. In one embodiment, the antioxidant compound increases the survival of motor neuron cells by reducing the toxicity of associated astrocytes.

In one embodiment, the antioxidant compound is selected from monomethyl fumarate (MMF), andrographolide, (6aS)-6-methyl-5,6,6a,7-tetrahydro-4H-dibenzo[de,g]quinoline-10,11-diol and riluzole.

In one embodiment, the antioxidant compound is (6aS)-6-methyl-5,6,6a,7-tetrahydro-4H-dibenzo[de,g]quinoline-10,11-diol.

In one embodiment, the disease is ALS caused by a C9orf72 mutation and the antioxidant compound is MMF, andrographolide or (6aS)-6-methyl-5,6,6a,7-tetrahydro-4H-dibenzo[de,g]quinoline-10,11-diol, suitably andrographolide.

In one embodiment, the disease is ALS caused by a SOD1 mutation and the antioxidant compound is (6aS)-6-methyl-5,6,6a,7-tetrahydro-4H-dibenzo[de,g]quinoline-10,11-diol or riluzole, suitably (6aS)-6-methyl-5,6,6a,7-tetrahydro-4H-dibenzo[de,g]quinoline-10,11-diol.

In one embodiment, the disease is ALS caused by a sporadic mutation and the antioxidant compound is MMF, andrographolide, riluzole, or (6aS)-6-methyl-5,6,6a,7-tetrahydro-4H-dibenzo[de,g]quinoline-10,11-diol, suitably andrographolide.

In another aspect, the invention provides for an in vitro method of screening a candidate therapeutic agent(s) for its ability to reduce the level of misfolded SOD1 protein in astrocytes, the method comprising:
1) exposing induced astrocytes derived from fibroblast stem cells to a candidate therapeutic;
2) comparing amounts of misfolded SOD1 between said induced astrocytes exposed to said candidate therapeutics and control cells.

In one embodiment, the control cells are induced astrocytes that are not exposed to said candidate therapeutic (unexposed induced astrocytes).

In one embodiment, the method may comprise comparing the amount of SOD1 aggregates between said induced astrocytes exposed to said candidate therapeutics and control cells. In one embodiment, the method may comprise comparing the amounts of SOD1 perinuclear aggregates between said induced astrocytes exposed to said candidate therapeutics and control cells.

In another aspect, the invention provides for an in vitro method of screening a candidate therapeutic agent(s) for its ability to increase motor neuron cell survival, the method comprising:
1) exposing motor neuron cells to a candidate therapeutic;
2) after a period of time, comparing the number of cells that survive between said motor neuron cells exposed to the candidate therapeutic and motor neuron cells exposed to a control.

In one embodiment, the period of time is between 1-5 days, suitably between 2-4 days, suitably 3 days.

In one embodiment, the motor neuron cells are in the presence of astrocytes. In one embodiment, the astrocytes and motor neuron cells are from a patient suffering from a neurodegenerative disease. In one embodiment the astrocytes and motor neuron cells are from a patient suffering from ALS.

The foregoing and other features and advantages of the invention will become more apparent from the following detailed description, which proceeds with reference to the accompanying drawings. Such description is meant to be illustrative, and not limiting, of the invention. Obvious variants of the disclosed (6a5)-6-methyl-5,6,6a,7-tetrahydro-4H-dibenzo[de,g]quinoline-10,11-diol crystalline complex in the text, including those described by the drawings and examples will be readily apparent to the person of ordinary skill in the art having the present disclosure, and such variants are considered to be a part of the current invention.

### BRIEF DESCRIPTION OF THE DRAWINGS

FIG. 1. Direct conversion of ALS patient fibroblasts into iNPCs. Fibroblasts are transduced using retroviral vectors containing the reprogramming factors Oct4, Sox2, Klf4 and c-Myc and supplemented with NPC medium and growth factors. Cells were grown until the 18-day mark where iNPCs were obtained.

FIG. 2. Quantification of mouse motor neuron rescue in co-cultures with induced astrocytes from healthy controls and ALS patients (CRT: pooled data from three healthy controls; ALS patients with C9orf72 mutations: C9orf72_183, C9orf72_78 and C9orf72_201; ALS patients with SOD1 mutations: SOD1_210, SOD1_102 and SOD1_100; sporadic ALS patients: sALS_17, sALS_12 and sALS_009). The change of motor neuron survival using 5 μM or 10 μM andrographolide and (6a5)-6-methyl-5,6,6a,7-tetrahydro-4H-dibenzo[de,g]quinoline-10,11-diol (labelled as Drug) were compared to vehicle (DMSO).

FIG. 3. Quantification of mouse motor neuron rescue in co-cultures with induced astrocytes from healthy controls and ALS patients (CRT: pooled data from three healthy controls; ALS patients with C9orf72 mutations: C9orf72_183, C9orf72_78 and C9orf72_201; ALS patients with SOD1 mutations: SOD1_210, SOD1_102 and SOD1_100; sporadic ALS patients: sALS_17, sALS_12 and sALS_009). The change of motor neuron survival using 5 μM or 10 μM monomethyl fumarate and riluzole were compared to vehicle (DMSO).

FIG. 4. Further data showing quantification of mouse Hb9GFP+ motor neuron rescue in co-cultures with induced astrocytes by an increase in percentage of motor neuron survival 3 days after administration of riluzole, andrographolide and (6aS)-6-methyl-5,6,6a,7-tetrahydro-4H-dibenzo[de,g]quinoline-10,11-diol (labelled as Drug) at 10uM compared to vehicle (DMSO). The human iAstrocytes were from the same various ALS patients: healthy controls (Control) and 3 different sporadic ALS patients (sALS, n=3); 3 different ALS patients with SOD1 mutations (SOD1, n=3) and 3 different ALS patients with C9orf72 mutations (C9orf, n=3). * p <0.05; ** p<0.01; *** p <0.001; **** p <0.0001.

FIG. 5. Quantification of human induced motor neuron rescue in co-cultures with induced astrocytes from healthy controls and ALS patients (healthy controls: CTR_155, CTR_3050 and CTR_209; ALS patients with C9orf72 mutations: C9orf72_183, C9orf72_78 and C9orf72_201; ALS patient with SOD1 mutation: SOD1_210; sporadic ALS patients: sALS_17, sALS_12 and sALS_009). The change of motor neuron survival using 10 μM andrographolide and (6aS)-6-methyl-5,6,6a,7-tetrahydro-4H-dibenzo[de,g]quinoline-10,11-diol (labelled as Drug) were compared to vehicle (DMSO).

FIG. 6. Quantification of human induced motor neuron rescue in co-cultures with induced astrocytes from healthy controls and ALS patients (healthy controls: CTR 155, CTR 3050 and CTR_209; ALS patients with C9orf72 mutations: C9orf72_183, C9orf72_78 and C9orf72_201; ALS patient with SOD1 mutation: SOD1_210; sporadic ALS patients: sALS_17, sALS_12 and sALS_009). The change of motor neuron survival using 10 μM monomethyl fumarate and riluzole were compared to vehicle (DMSO).

FIG. 7. Reduced SOD1 misfolding by andrographolide and (6aS)-6-methyl-5,6,6a,7-tetrahydro-4H-dibenzo[de,g]quinoline-10,11-diol (labelled as Drug) in human iAstrocytes. The iAstrocytes were from various ALS patients (healthy controls: CTR_3050, CTR_155 and CTR_AG; ALS patients with C9orf72 mutations: C9orf72_78, C9orf72_183 and C9orf72_201; ALS patients with SOD1 mutations: SOD1_100, SOD1_102 and SOD1_ND; sporadic ALS patients: sALS_009 and sALS_17).

FIG. 8. Reduced SOD1 misfolding by monomethyl fumarate and riluzole in human iAstrocytes. The iAstrocytes were from various ALS patients (healthy controls: CTR_3050, CTR_155 and CTR_AG; ALS patients with C9orf72 mutations: C9orf72_78, C9orf72_183 and C9orf72_201; ALS patients with SOD1 mutations: SOD1_100, SOD1_102 and SOD1_ND; sporadic ALS patients: sALS_009 and sALS_17).

FIG. 9. Further data showing reduction in SOD1 misfolding by a reduction in percentage of misfolded SOD1 perinuclear aggregates after administration of (6a5)-6-methyl-5,6,6a,7-tetrahydro-4H-dibenzo[de,g]quinoline-10,11-diol (labelled as Drug) at 10uM to human iAstrocytes for 48 hours. The iAstrocytes were from the same various ALS patients: healthy controls (CTR n=3), 3 different sporadic ALS patients (sALS, n=3); 3 different ALS patients with SOD1 mutations (SOD1, n=3) and 3 different ALS patients with C9orf72 mutations (C9orf, n=3). DMSO treatment condition for each individual donor is considered 100% and treatment with (6a5)-6-methyl-5,6,6a,7-tetrahydro-4H-dibenzo[de,g]quinoline-10,11-diol is a percentage of that value. We report reduction of misfolded SOD1 in all patient lines treated, with significant reduction in misfolded SOD1 in 6 different patient lines. * p <0.05.

### DETAILED DESCRIPTION OF THE PREFERRED EMBODIMENTS

The term ‘(6aR)-6-methyl-5,6,6a,7-tetrahydro-4H-dibenzo[de,g]quinoline-10,11-diol’ means R-(−)-10,11-dihydroxyaporphine, including prodrug, salts, solvates, hydrates, and co-crystals thereof.

The term ‘(6a5)-6-methyl-5,6,6a,7-tetrahydro-4H-dibenzo[de,g]quinoline-10,11-diol’ means S-(+)-10,11-dihydroxyaporphine, including prodrug, salts, solvates, hydrates, and co-crystals thereof.

The term ‘6-methyl-5,6,6a,7-tetrahydro-4H-dibenzo[de,g]quinoline-10,11-diol’ means (6aR)-6-methyl-5,6,6a,7-tetrahydro-4H-dibenzo[de,g]quinoline-10,11-diol, or (6aS)-6-methyl-5,6,6a,7-tetrahydro-4H-dibenzo[de,g]quinoline-10,11-diol, or racemic form of (6aR)-6-methyl-5,6,6a,7-tetrahydro-4H-dibenzo[de,g]quinoline-10,11-diol and (6a5)-6-methyl-5,6,6a,7-tetrahydro-4H-dibenzo[de,g]quinoline-10,11-diol, including prodrug, salts, solvates, hydrates, and co-crystals thereof.

As used herein, the terms ‘treat’, ‘treating’ or ‘treatment’ means to alleviate, reduce or abrogate one or more symptoms or characteristics of a disease and may be curative, palliative, prophylactic or slow the progression of the disease.

The term “effective amount” means an amount that will result in a desired effect or result, e.g., reducing the frequency of SOD1 protein misfolding, reducing the accumulation of SOD1 misfolded protein, or reducing aggregation of SOD1 protein. The term ‘therapeutically effective amount’ means an amount of (6aS)-6-methyl-5,6,6a,7-tetrahydro-4H-dibenzo[de,g]quinoline-10,11-diol, alone or combined with other active ingredients, that will elicit a desired biological or pharmacological response, e.g., effective to prevent, alleviate, or ameliorate symptoms of a disease or disorder; slow, halt or reverse an underlying disease process or progression; partially or fully restore cellular function; or prolong the survival of the subject being treated.

The term ‘patient’ or ‘subject’ includes mammals, including non-human animals and especially humans. In one embodiment the patient or subject is a human. In another embodiment the patient or subject is a human male. In another embodiment the patient or subject is a human female.

The term ‘significant’ or ‘significantly’ is determined by t-test at 0.05 level of significance.

The present invention relates to methods of using of (6aS)-6-methyl-5,6,6a,7-tetrahydro-4H-dibenzo[de,g]quinoline-10,11-diol to reduce the frequency of SOD1 protein misfolding, reduce the accumulation of SOD1 misfolded protein, or reduce aggregation of SOD1 protein in a cell, tissue or animal.

The present invention further relates to methods of using (6aS)-6-methyl-5,6,6a,7-tetrahydro-4H-dibenzo[de,g]quinoline-10,11-diol for the treatment, prevention, alleviation, or amelioration of a disease that is mediated by SOD1 protein misfolding or accumulation of misfolded SOD1 protein. The present invention further relates to method of using (6aS)-6-methyl-5,6,6a,7-tetrahydro-4H-dibenzo[de,g]quinoline-10,11-diol for extending/increasing the longevity of a cell, tissue, organ, or animal.

Accordingly, in one aspect, the present invention provides for a method of reducing the level of misfolded SOD1 in a cell, comprising the step of contacting said cell with an effective amount of (6a5)-6-methyl-5,6,6a,7-tetrahydro-4H-dibenzo[de,g]quinoline-10,11-diol.

In one embodiment, the method may be an in vitro method.

In a related aspect, the present invention provides for a method of increasing the level of properly folded SOD1 in a cell, comprising the step of contacting said cell with an effective amount of (6a5)-6-methyl-5,6,6a,7-tetrahydro-4H-dibenzo[de,g]quinoline-10,11-diol.

In one embodiment, the method may be an in vitro method.

In another aspect, the present invention provides for a method of: (a) reducing SOD1 protein misfolding in a cell, in terms of frequency or rate at which SOD1 protein misfolding occurs, (b) reducing accumulation of misfolded SOD1 protein in a cell, or (c) reducing SOD1 protein aggregation in a cell, said method comprising the step of contacting said cell with an effective amount of (6aS)-6-methyl-5,6,6a,7-tetrahydro-4H-dibenzo[de,g]quinoline-10,11-diol.

In one embodiment, the method may be an in vitro method

In another aspect, the invention provides for a method of increasing cell lifespan, comprising the step of contacting said cell with an effective amount of (6aS)-6-methyl-5,6,6a,7-tetrahydro-4H-dibenzo[de,g]quinoline-10,11-diol.

In one embodiment, the method may be an in vitro method

In one embodiment, the cell in one of the above aspects, or other aspect or embodiments herein, is a cell type or from a tissue selected from any one or more of: adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder and vagina. In a further embodiment, said brain cell is from a brain tissue selected from cerebrum (including cerebral cortex, basal ganglia (often called the striatum), and olfactory bulb), cerebellum (including dentate nucleus, interposed nucleus, fastigial nucleus, and vestibular nuclei), diencephalon (including thalamus, hypothalamus, etc. and the posterior portion of the pituitary gland), and brain-stem (including pons, substantia nigra, medulla oblongata). In a further embodiment, said brain cell is selected from a neuron or glia cell (e.g., an astrocyte, oligodendrocyte, or microglia). In a further embodiment, said neuron is a sensory neuron, motor neuron, interneuron, or brain neuron.

In one embodiment, the cell is an animal cell, e.g., mammalian cell. In a further embodiment, said cell in a human cell or non-human cell. In a further embodiment, said cell is a human cell. In a further embodiment, said cell is in vitro, in vivo, or ex vivo.

In another embodiment, the cell is a diseased cell. In another embodiment, the cell is diseased cell from a patient suffering from a disease or disorder disclosed herein.

In another aspect, the invention provides for a method of treating an animal having a disease or disorder would benefit from reducing the frequency of SOD1 protein misfolding, reducing the accumulation of SOD1 misfolded protein, or reducing aggregation of SOD1 protein, for example, where a symptom that is prevented, alleviated, or ameliorated, or a disease process or progression that slowed, halted or reversed, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides for (6aS)-6-methyl-5,6,6a,7-tetrahydro-4H-dibenzo[de,g]quinoline-10,11-diol for use in the treatment of a disease or disorder by reducing the frequency of SOD1 protein misfolding, reducing the accumulation of SOD1 misfolded protein, or reducing aggregation of SOD1 protein, for example, where a symptom that is prevented, alleviated, or ameliorated, or a disease process or progression that slowed, halted or reversed.

In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising (6aS)-6-methyl-5,6,6a,7-tetrahydro-4H-dibenzo[de,g]quinoline-10,11-diol may be for administration to the animal in an effective amount.

In one embodiment, the animal is mammal. In a further embodiment, the mammal is a human. In another embodiment, the mammal is a non-human mammal.

In another embodiment, said disease or disorder is caused by SOD1 protein misfolding, accumulation of misfolded SOD1 protein, or SOD1 protein aggregation.

In another embodiment, said disease is selected from any one or more of: age-related macular degeneration, Alzheimer's disease (AD), amyotrophic lateral sclerosis (ALS), atherosclerosis, autism spectrum disorder (ASD), benign focal amyotrophy, cerebral infarction, Creutzfeldt-Jakob disease Crohn's disease, Duchenne's paralysis, Friedreich's ataxia, frontotemporal dementia (FTD), glaucoma, hereditary spastic paraplegia (HSP), Huntington's disease (HD), Inclusion Body Myopathy (IBM)inflammatory bowel disease, ischemia, Kugelberg-Welander syndrome, Lewy body diseases (LBD), Lou Gehrig's disease, multiple sclerosis (MS), myocardial infarction, necrotizing enterocolitis, Neurofibromatosis type I, Paget's disease of the bone (PDB), Parkinson disease (PD), primary lateral sclerosis (PLS), progressive bulbar palsy (PBP), progressive muscular atrophy (PMA), pseudobulbar palsy, spinal muscular atrophy (SMA), ulcerative colitis, Valosin-Containing Protein (VCP)-related disorders, or Werdnig-Hoffmann disease, transient ischemic attack, ischaemia, cerebral hemorrhage, senile cataract, retinal ischemia, retinal vasculitis, Brown-Vialetto-Van Laere syndrome, Eales Disease, meningitis and encephalitis, post-traumatic stress disorder, Charcot-Marie-Tooth Disease, macular degeneration, X-Linked Bulbo-Spinal Atrophy, presenile dementia, depressive disorder, temporal lobe epilepsy, Hereditary Leber Optic Atrophy, cerebrovascular accident, subarachnoid hemorrhage, and schizophrenia.

In another embodiment, said disease is a neurological disease.

In one embodiment, the disease is a neurodegenerative disease or disorder.

In one embodiment, the disease is ALS.

In one embodiment, the disease is ALS caused by a mutation. In one embodiment, the disease is ALS caused by a mutation selected from: a C9orf72 mutation, a SOD1 mutation, or a sporadic mutation. In one embodiment, the disease is ALS caused by a SOD1 mutation.

In another aspect, the invention provides for a method of increasing lifespan or treating a disease or disorder resulting in accelerated aging or other abnormal aging process in an animal, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides for (6aS)-6-methyl-5,6,6a,7-tetrahydro-4H-dibenzo[de,g]quinoline-10,11-diol for use in the treatment of a disease or disorder resulting in accelerated aging or other abnormal aging process in an animal.

In another aspect, the invention provides for (6aS)-6-methyl-5,6,6a,7-tetrahydro-4H-dibenzo[de,g]quinoline-10,11-diol for use in the treatment of a disease or disorder by increasing lifespan of an animal.

In one embodiment, the disease or disorder is premature ageing. In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising (6aS)-6-methyl-5,6,6a,7-tetrahydro-4H-dibenzo[de,g]quinoline-10,11-diol may be for administration to the animal in an effective amount.

In one embodiment, said animal is a mammal. In another embodiment, said mammal is a human or a non-human mammal.

In a related aspect, the invention provides for a method of treating premature aging due to chemical or radiation exposure. In one embodiment, the premature aging is due to exposure to chemotherapy, radiation therapy, or UV radiation. In a further embodiment, the UV radiation is artificial, e.g., tanning bed, or solar UV radiation, i.e., sun exposure.

In a related aspect, the invention provides for (6aS)-6-methyl-5,6,6a,7-tetrahydro-4H-dibenzo[de,g]quinoline-10,11-diol for use in the treatment of premature aging due to chemical or radiation exposure.

In one embodiment, the premature aging is due to exposure to chemotherapy, radiation therapy, or UV radiation.

In one embodiment, the UV radiation is artificial, e.g., tanning bed, or solar UV radiation, i.e., sun exposure.

In one embodiment, (6aS)-6-methyl-5,6,6a,7-tetrahydro-4H-dibenzo[de,g]quinoline-10,11-diol may be comprised in a pharmaceutical composition.

In one embodiment, (6aS)-6-methyl-5,6,6a,7-tetrahydro-4H-dibenzo[de,g]quinoline-10,11-diol or pharmaceutical composition comprising may be for administration to the animal in an effective amount.

The present invention further provides of the use of (6aS)-6-methyl-5,6,6a,7-tetrahydro-4H-dibenzo[de,g]quinoline-10,11-diol for the preparation of a medicament for treating a human having any one of the diseases or disorders disclosed herein or for use in any method of the present invention involving the administration of (6aS)-6-methyl-5,6,6a,7-tetrahydro-4H-dibenzo[de,g]quinoline-10,11-diol to a human.

In another aspect, the invention provides for an in vitro method of screening a candidate therapeutic agent(s) for its ability to reduce the level of misfolded SOD1 protein in astrocytes, the method comprising the steps of:

(a) exposing induced astrocytes derived from fibroblast stem cells to said candidate therapeutic;

(b) comparing amounts of misfolded SOD1 between said induced astrocytes exposed to said candidate therapeutic and control cells.

In one embodiment, the control cells are induced astrocytes that are not exposed to said candidate therapeutic (i.e., unexposed induced astrocytes).

In one embodiment, the method may comprise comparing the amounts of SOD1 aggregates between said induced astrocytes exposed to said candidate therapeutics and control cells. In one embodiment, the method may comprise comparing the amounts of SOD1 perinuclear aggregates between said induced astrocytes exposed to said candidate therapeutics and control cells.

In another aspect, the invention provides for an in vitro method of screening a candidate therapeutic agent(s) for its ability to increase motor neuron cell survival, the method comprising:
3) exposing motor neuron cells to a candidate therapeutic;
4) comparing the number of cells that survive after a period of time between said motor neuron cells exposed to the candidate therapeutic and motor neuron cells exposed to a control.

In one embodiment, the period of time is between 1-5 days, suitably between 2-4 days, suitably 3 days.

In one embodiment, the motor neuron cells are in the presence of astrocytes. In one embodiment, the astrocytes and motor neuron cells are from a patient suffering from a neurodegenerative disease. In one embodiment the astrocytes and motor neuron cells are from a patient suffering from ALS.

In another aspect, the invention provides for a method of improving the survival of cells by reducing the toxicity of astrocytes.

In one embodiment, the cell is an animal cell, e.g., mammalian cell. In a further embodiment, said cell in a human cell or non-human cell. In a further embodiment, said cell is in vitro, in vivo, or ex vivo.

In another embodiment, the cell is a diseased cell. In another embodiment, the cell is diseased cell from a patient suffering from a neurodegenerative disease. In another embodiment, the cell is a diseased cell from a patient suffering from ALS.

In a further embodiment, the astrocytes are associated with the cells. In one embodiment, the astrocytes are from the same source as the cells. In one embodiment, the astrocytes are from a patient suffering from a neurodegenerative disease. In one embodiment, the astrocytes are from a patient suffering ALS.

In another embodiment, the cell is a motor neuron cell. In another embodiment, the cell is motor neuron cell from a patient suffering from a neurodegenerative disease. In another embodiment, the cell is a motor neuron cell from a patient suffering from ALS.

In another embodiment, the cell is a diseased motor neuron cell from a patient suffering from a neurodegenerative disease. In another embodiment, the cell is a diseased motor neuron cell from a patient suffering from ALS.

In another aspect, the invention provides a method of improving cell survival by reducing astrocyte toxicity in a cell, comprising a step of contacting the cell with an effective amount of an antioxidant compound

In another aspect, the invention provides a method of improving cell survival by reducing astrocyte toxicity in a cell, comprising a step of contacting the cell with an effective amount of (6aS)-6-methyl-5,6, 6a,7-tetrahydro-4H-dibenzo[de,g]quinoline-10,11-diol.

In another aspect, the invention provides a method of treating an animal having a disease or disorder that would benefit from reducing astrocyte toxicity or improving cell survival, the method comprising the step of administering a therapeutically effective amount of a pharmaceutical composition comprising an antioxidant compound to said animal.

In another aspect, the invention provides a method of treating an animal having a disease or disorder that would benefit from reducing astrocyte toxicity or improving cell survival, the method comprising the step of administering a therapeutically effective amount of a pharmaceutical composition comprising (6aS)-6-methyl-5,6,6a,7-tetrahydro-4H-dibenzo[de,g]quinoline-10,11-diol to said animal.

In another aspect, the invention provides an antioxidant compound for use in the treatment of a disease or disorder by reducing the toxicity of astrocytes and/or by increasing the survival of cells.

In one embodiment, the disease or disorder is a neurodegenerative disease or disorder, for example any of those listed hereinabove. In one embodiment, the disease or disorder is ALS.

In one embodiment, the cells are motor neuron cells.

In one embodiment, the astrocytes are associated with the cells.

In one embodiment, the antioxidant compound increases the survival of cells by reducing the toxicity of astrocytes. In one embodiment, the antioxidant compound increases the survival of motor neuron cells by reducing the toxicity of associated astrocytes.

In one embodiment, the antioxidant compound is selected from monomethyl fumarate (MMF), andrographolide, (6aS)-6-methyl-5,6,6a,7-tetrahydro-4H-dibenzo[de,g]quinoline-10,11-diol and riluzole.

In one embodiment, the antioxidant compound is (6aS)-6-methyl-5,6,6a,7-tetrahydro-4H-dibenzo[de,g]quinoline-10,11-diol.

In one embodiment, the disease is ALS caused by a C9orf72 mutation and the antioxidant compound is monomethyl fumarate (MMF), andrographolide or (6aS)-6-methyl-5,6,6a,7-tetrahydro-4H-dibenzo[de,g]quinoline-10,11-diol, suitably andrographolide.

In one embodiment, the disease is ALS caused by a SOD1 mutation and the antioxidant compound is (6aS)-6-methyl-5,6,6a,7-tetrahydro-4H-dibenzo[de,g]quinoline-10,11-diol or riluzole, suitably (6aS)-6-methyl-5,6,6a,7-tetrahydro-4H-dibenzo[de,g]quinoline-10,11-diol.

In one embodiment, the disease is ALS caused by a sporadic mutation and the antioxidant compound is MMF, andrographolide, riluzole, or (6aS)-6-methyl-5,6,6a,7-tetrahydro-4H-dibenzo[de,g]quinoline-10,11-diol, suitably andrographolide.

The pharmaceutical compositions of the present invention comprise a therapeutically effective amount (6aS)-6-methyl-5,6,6a,7-tetrahydro-4H-dibenzo[de,g]quinoline-10,11-diol and at least one pharmaceutically acceptable excipient. The term “excipient” refers to a pharmaceutically acceptable, inactive substance used as a carrier for the pharmaceutically active ingredient ((6aS)-6-methyl-5,6,6a,7-tetrahydro-4H-dibenzo[de,g]quinoline-10,11-diol), and includes antiadherents, binders, coatings, disintegrants, fillers, diluents, solvents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for injection or infusion can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, including autoinjectors, or multiple dose vials made of glass or plastic.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid solution, suspension, emulsion or other liquid form, injectable suspension, solution, emulsion, etc., suppository, or topical or transdermal preparation or patch. The pharmaceutical formulations generally contain about 1% to about 99% by weight of (6aS)-6-methyl-5,6,6a,7-tetrahydro-4H-dibenzo[de,g]quinoline-10,11-diol and 99% to 1% by weight of a suitable pharmaceutical excipient. In one embodiment, the dosage form is an oral dosage form. In another embodiment, the dosage form is a parenteral dosage form. In another embodiment, the dosage form is an enteral dosage form. In another embodiment, the dosage form is a topical dosage form. In one embodiment, the pharmaceutical dosage form is a unit dose. The term ‘unit dose’ refers to the amount of (6aS)-6-methyl-5,6,6a,7-tetrahydro-4H-dibenzo[de,g]quinoline-10,11-diol administered to a patient in a single dose.

In some embodiments, a pharmaceutical composition of the present invention is delivered to a subject via a parenteral route, an enteral route, or a topical route.

Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories. Oral administration includes sublingual and buccal administration.

Topical administration includes administration to a body surface, such as skin or mucous membranes, including intranasal and pulmonary administration. Transdermal forms include cream, foam, gel, lotion or ointment. Intranasal and pulmonary forms include liquids and powders, e.g., liquid spray.

The dose may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy.

In one embodiment, the daily dose of (6aS)-6-methyl-5,6,6a,7-tetrahydro-4H-dibenzo[de,g]quinoline-10,11-diol administered to a patient is selected from: up to 200 mg, 175 mg, 150 mg, 125 mg, 100 mg, 90 mg, 80 mg, 70 mg, 60 mg, 50 mg, 30 mg, 25 mg, 20 mg, 15 mg, 14 mg, 13 mg, 12 mg, 11 mg, 10 mg, 9 mg, 8 mg, 7 mg, 6 mg, 5 mg, 4 mg, 3 mg, or up to 2 mg. In another embodiment, the daily dose is at least 1 mg, 2 mg, 3 mg, 4 mg, 5 mg, 6 mg, 7 mg, 8 mg, 9 mg, 10 mg, 12 mg, 13 mg, 14 mg, 15 mg, 20 mg, 25 mg, 30 mg, 40 mg, 50 mg, 60 mg, 70 mg, 80 mg, 90 mg, 100 mg, 125 mg, 150 mg, 175 mg, 200 mg, 300 mg, 400 mg, 500 mg, 600 mg, 700 mg, 800 mg, 900 mg, 1,000 mg, 2,000 mg, 3,000 mg, 4,000 mg, or at least 5,000 mg. In another embodiment, the daily dose is 1-2 mg, 2-4 mg, 1-5 mg, 5-7.5 mg, 7.5-10 mg, 10-15mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg, 50-75 mg, 75-100 mg, 100-125 mg, 125-150 mg, 150-175 mg, 175-200 mg, 5-200 mg, 5-300 mg, 5-400 mg, 5-500 mg, 5-600 mg, 5-700 mg, 5-800 mg, 5-900 mg, 5-1,000 mg, 5-2,000 mg, 5-5,000 mg or more than 5,000 mg.

In another embodiment, a single dose of (6aS)-6-methyl-5,6,6a,7-tetrahydro-4H-dibenzo[de,g]quinoline-10,11-diol administered to a patient is selected from: 1 mg, 2 mg, 3 mg, 4 mg, 5 mg, 6 mg, 7 mg, 8 mg, 9 mg, 10 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 100 mg, 110 mg, 120 mg, 130 mg, 140 mg ,150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg 490 mg, 500 mg, 600 mg, 700 mg, 800 mg, 900 mg, 1,000 mg, 2,000 mg, 3,000 mg, 4,000 mg, or 5,000 mg. In another embodiment, a single dose of (6aS)-6-methyl-5,6,6a,7-tetrahydro-4H-dibenzo[de,g]quinoline-10,11-diol administered to a patient is selected from: 1-2 mg, 2-4 mg, 1-5 mg, 5-7.5 mg, 7.5-10 mg, 10-15mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, 45-50 mg, 50-75 mg, 75-100 mg, 100-125 mg, 125-150 mg, 150-175 mg, 175-200 mg, 200-225 mg, 225-250 mg, 250-275 mg, 275-300 mg, 300-325 mg, 325-350 mg, 350-375 mg, 375-400 mg, 400-425 mg, 425-450 mg, 450-475 mg, 475-500 mg, 500-1,000 mg, 1,000-2,000 mg, 3,000-4,000 mg, 4,000-5,000 mg, or more than 5,000 mg. In one embodiment, the single dose is administered by a route selected from any one of: oral, buccal, or sublingual administration. In another embodiment, said single dose is administered by injection, e.g., subcutaneous, intramuscular, or intravenous. In another embodiment, said single dose is administered by inhalation or intranasal administration.

As a non-limited example, the dose of (6aS)-6-methyl-5,6,6a,7-tetrahydro-4H-dibenzo[de,g]quinoline-10,11-diol administered by subcutaneous injection may be about 3 to 5,000 mg per day to be administered in divided doses. A single dose of (6aS)-6-methyl-5,6,6a,7-tetrahydro-4H-dibenzo[de,g]quinoline-10,11-diol administered by subcutaneous injection may be about 1-6 mg, preferably about 1-4 mg, 1-3 mg, or 2 mg. Other embodiments include ranges of about 5-5,000 mg, preferably about 100-1,000 mg, 100-500 mg, 200-400 mg, 250-350 mg, or 300 mg. Subcutaneous infusion may be preferable in those patients requiring division of injections into more than 10 doses daily. The continuous subcutaneous infusion dose may be 1 mg/hour daily and is generally increased according to response up to 4 mg/hour.

The fine particle dose of (6a5)-6-methyl-5,6,6a,7-tetrahydro-4H-dibenzo[de,g]quinoline-10,11-diol administered by pulmonary administration, e.g., inhalation using a pressurized metered dose inhaler (pMDI), dry powder inhaler (DPI), soft-mist inhaler, nebulizer, or other device, may be in the range of about, 0.5-15 mg, preferably about 0.5-8 mg or 2-6 mg. Other embodiments include ranges of about 5-5,000 mg, preferably about 100-1,000 mg, 100-500 mg, 200-400 mg, 250-350 mg, or 300 mg. The Nominal Dose (ND), i.e., the amount of drug metered in the receptacle (also known as the Metered Dose), of (6aS)-6-methyl-5,6,6a,7-tetrahydro-4H-dibenzo[de,g]quinoline-10,11-diol administered by pulmonary administration may be, for example, in the range of 0.5-15 mg, 3-10 mg, 10-15mg, 10-12.5 mg, 12.5-15 mg, 15-17.7 mg, 17.5-20 mg, 20-25 mg, 20-22.5 mg, 22.5-25 mg, 25-30 mg, 25-27.5 mg, 27.5-30 mg, 30-35 mg, 35-40 mg, 40-45 mg, or 45-50 mg. Other embodiments include ranges of about 5-5,000 mg, preferably about 100-1,000 mg, 100-500 mg, 200-400 mg, 250-350 mg, or 300 mg. Long-acting pharmaceutical compositions may be administered, 1, 2, 3, 4, 5, 6, 7, 8, 9, 10 or more than 10 times daily (preferably <10 times per day), every other day, every 3 to 4 days, every week, or once every two weeks depending on half-life and clearance rate of the particular formulation.

### EXAMPLES

The following examples illustrate the invention without intending to limit the scope of the invention.

### Example 1

Over the last decade, in vitro modelling of neurodegeneration has undergone impressive development, mainly due to the reprogramming of adult human fibroblasts into induced pluripotent stem cells (iPSCs) and induced neural progenitor cells (iNPCs). In the ALS research field, this offers an opportunity to model familial and sporadic diseases in vitro.

NPCs harvested from post mortem spinal cord of ALS patients have already been successfully differentiated into motor neurons, astrocytes and oligodendrocytes. Deriving astrocytes using this method avoids inducing major epigenetic alterations. However, the availability of post-mortem samples is limited. In addition, the disadvantages of reprogramming astrocytes from human derived iPSCs include time-consuming protocols, as well as complex and highly-variable maturation time of the astrocytes.

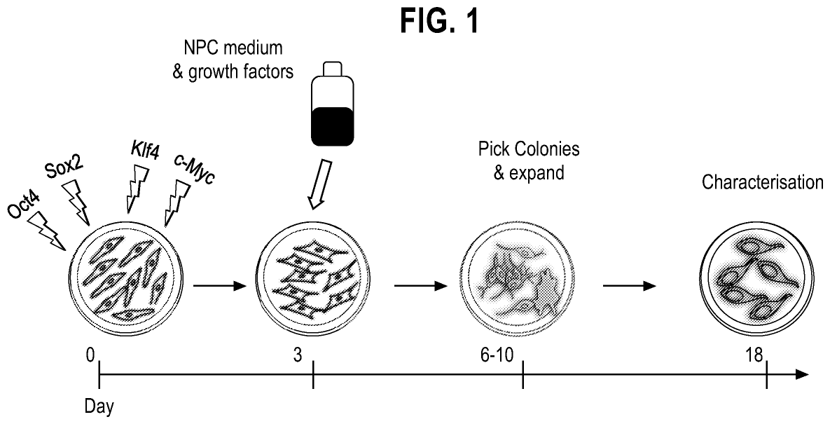

Therefore, a promising alterative to iPSC resources is the direct reprogramming of fibroblasts into astrocytes from an immuno-matched host. Instead of generating iPSCs, direct reprogramming involves the use of cell-lineage transcription factors to convert adult somatic cells into another cell type. This technology has been used to generate sub-specific neural lineages such as cholinergic, dopaminergic and motor neurons. Direct reprogramming technology was also used to derive astrocytes from ALS patient fibroblasts, and tripotent iNPCs from ALS patients and controls were generated within one month. When these cells were differentiated into astrocytes, they displayed similar toxicity towards motor neurons in co-cultures as autopsy-derived astrocytes, making them useful tools in the development of drug screens (FIG. 1).

Methodology:

iNPCs were generated from adult human fibroblasts from patients who had been diagnosed with ALS and from age-matched healthy controls, using an approach reported previously (Kim et al PNAS, 2001. 108(19), 7838-7843; Meyer et al., PNAS, 2014. 111(2), 829-832). iNPCs are differentiated into induced astrocytes (iAstrocytes) by culturing the progenitors in iAstrocyte medium for a total of 7 days with a medium change at day 3.

Induced astrocytes from control or ALS patients were used in a co-culture assay to determine their effect on mouse motor neuron (MN) survival. Mouse embryonic stem cell-derived motor neurons expressing green fluorescence protein (GFP) under the control of the HB9 promoter were sorted and added to iAstrocytes from patients and controls. Meanwhile, andrographolide, (6aS)-6-methyl-5,6,6a,7-tetrahydro-4H-dibenzo[de,g]quinoline-10,11-diol, monomethyl fumarate (MMF) and Riluzole were screened in this co-culture system of patient iAstrocytes and wildtype mouse MNs. The survival of mouse MNs was monitored on Day 1 and 3 with confocal image acquisition.

Result:

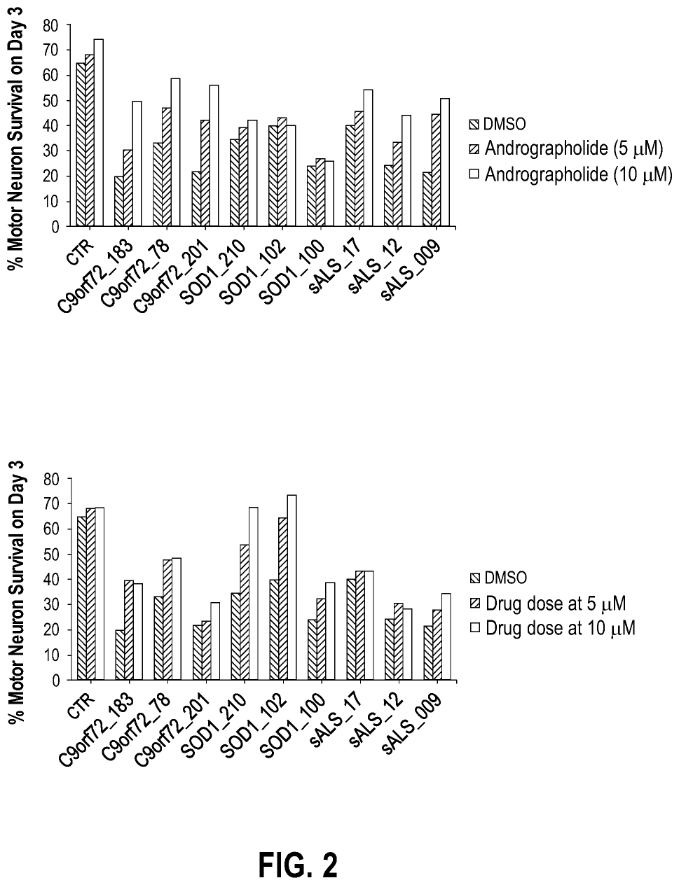

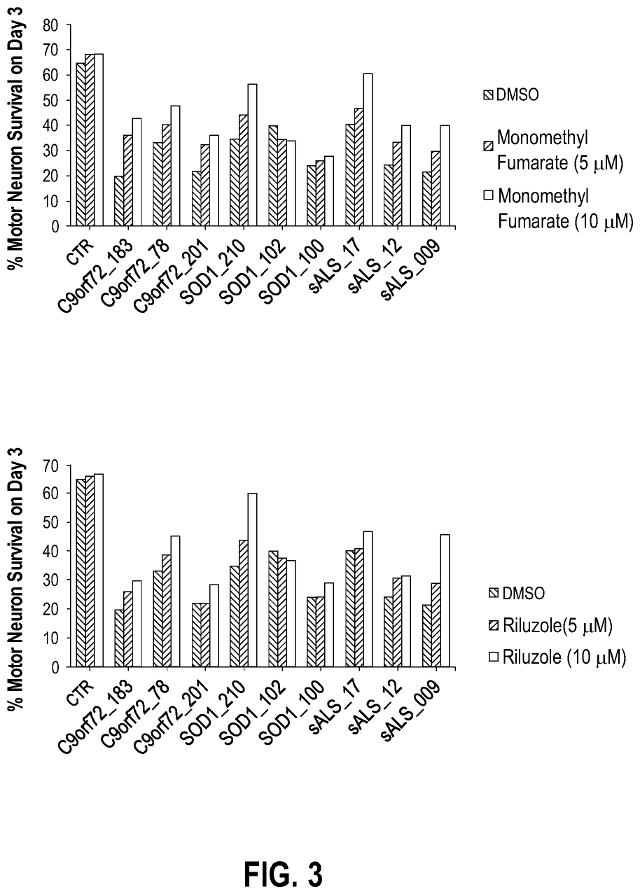

The MN survival on Day 3 was evaluated as a percentage of survived MN cells observed on Day 1. As expected, iAstrocytes from a healthy control did not significantly change the survival of mouse MNs on Day 3. The introduction of all four drugs also did not change the survival of mouse MNs (FIGS. 2 and 3).

When iAstrocytes from three ALS patient with C9orf72 mutation (i.e., patient C9orf72_183, C9orf72_201 and C9orf72_78) were co-cultured with mouse MNs, no more than 33% of the MN cells survived on Day 3, among all three ALS patients. However, the survival of MN cells on Day 3 was significantly improved, when andrographolide, (6aS)-6-methyl-5,6,6a,7-tetrahydro-4H-dibenzo[de,g]quinoline-10,11-diol and MMF were introduced to the culture. More specifically, (6aS)-6-methyl-5,6,6a,7-tetrahydro-4H-dibenzo[de,g]quinoline-10,11-diol has improved the MN survival to up to 38%.

When iAstrocytes from ALS patients with SOD1 mutation (i.e., patient SOD1_210, SOD1_102, SOD1 100) were co-cultured with mouse MNs, approximately 40% or less of the MN cells survived on Day 3. The survival of MN cells on Day 3 showed most significant improvement with the introduction of (6aS)-6-methyl-5,6,6a,7-tetrahydro-4H-dibenzo[de,g]quinoline-10,11-diol.

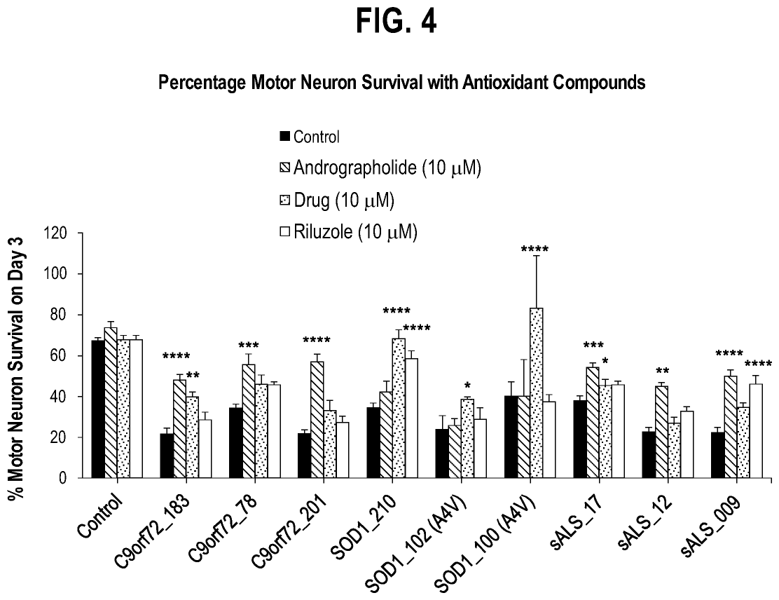

When iAstrocytes from three ALS patients with sALS mutation (i.e., patient sALS_17, patient sALS_12, patient sALS_009) were co-cultured with mouse MN, the survival of MN cells on Day 3 varied between 21 to 40%. In this study, the survival of MN cells on Day 3 was most significantly improved in the presence of andrographolide (FIGS. 2, 3 and 4).

### Example 2

Induced Astrocytes from healthy controls or ALS patients were also used in a co-culture assay to determine their effect on the survival of induced MN cells from the same healthy controls or ALS patients.

Methodology:

The preparation of iAstrocytes and induced MN cells has been described in Example 1. Similarly, andrographolide, (6a5)-6-methyl-5,6,6a,7-tetrahydro-4H-dibenzo[de,g]quinoline-10,11-diol, MMF and Riluzole were screened in this co-culture system. The MN survival on Day 3 was evaluated as a percentage of survived MN cells observed on Day 1.

Result:

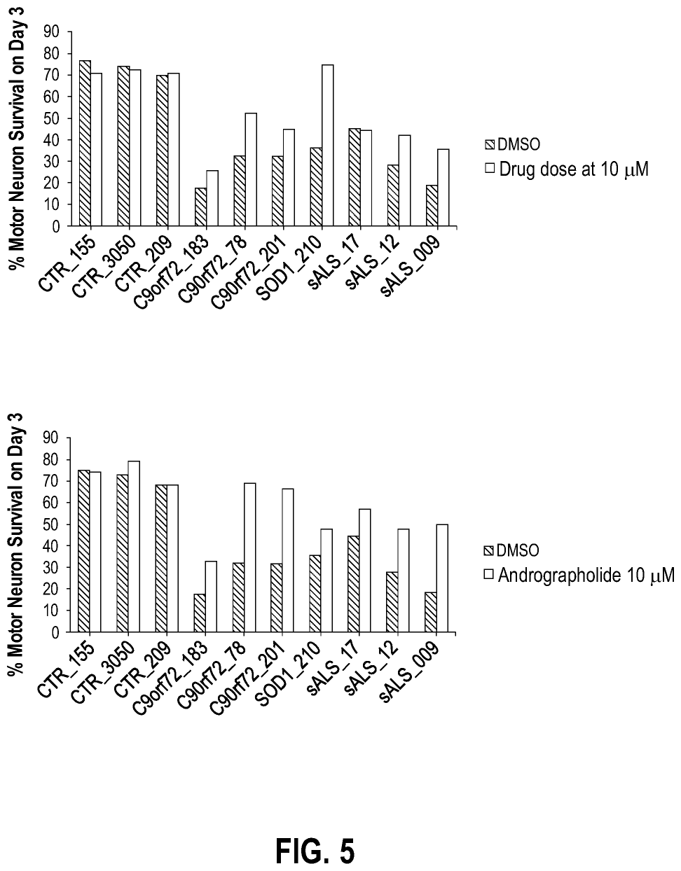

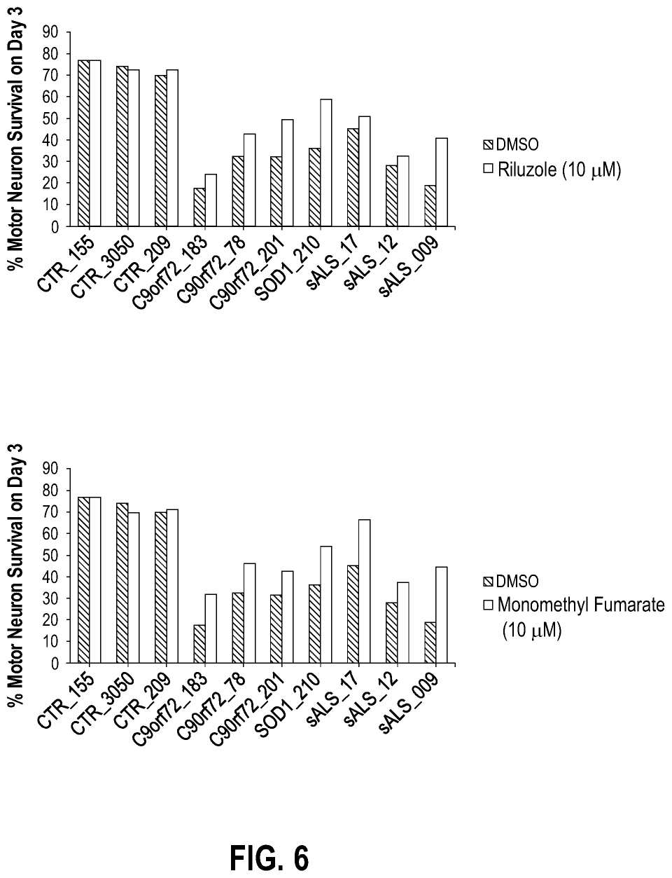

As expected, iAstrocytes from healthy controls did not significantly change the survival of induced MNs from the same healthy controls on Day 3. Also, the introduction of all four drugs also did not change the survival of human MNs (FIGS. 5 and 6).

When iAstrocytes from an ALS patient with C9orf72 mutation was co-cultured with induced MNs from the same ALS patient, no more than than 32% of human MN cells survived on Day 3. All four drugs showed some evidence to improve the MN survival at 10 μM, while andrographolide and (6aS)-6-methyl-5,6,6a,7-tetrahydro-4H-dibenzo[de,g]quinoline-10,11-diol exhibited the most significant outcome.

When iAstrocytes from an ALS patient with SOD1 mutation was co-cultured with induced MNs from the same patient, approximately 36% of the MN cells survived on Day 3. Among all drugs evaluated, (6aS)-6-methyl-5,6,6a,7-tetrahydro-4H-dibenzo[de,g]quinoline-10,11-diol most effectively dampened the toxicity of SOD1-derived astrocytes.

When iAstrocytes from an ALS patient with sALS mutation was co-cultured with induced MNs from the same patient, the survival of MN cells on Day 3 varied between 19 to 45%. In this study, all four drugs showed some evidence to improve the MN survival at 10 μM. In addition, the outcome of this study showed that (6a5)-6-methyl-5,6,6a,7-tetrahydro-4H-dibenzo[de,g]quinoline-10,11-diol and other drugs were beneficial at reducing toxicity caused by iAstrocytes from some sporadic patients over others, indicating the potential for a personalized medicine approach.

### Example 3

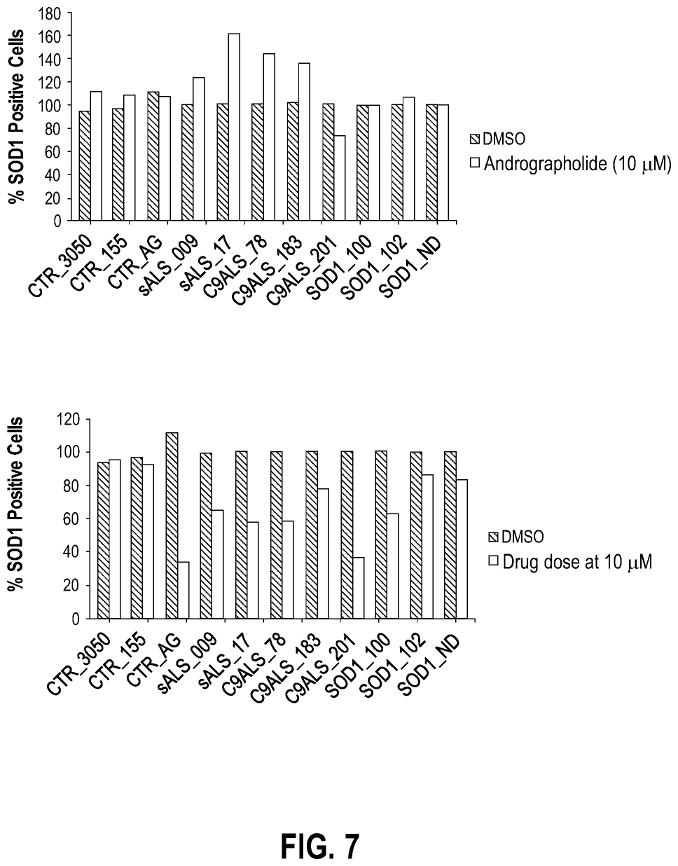

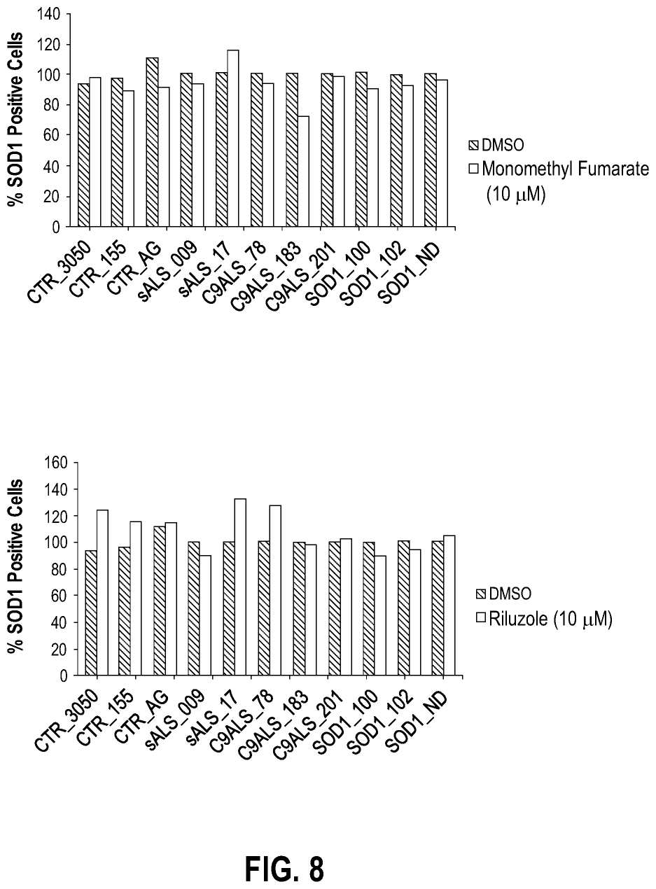

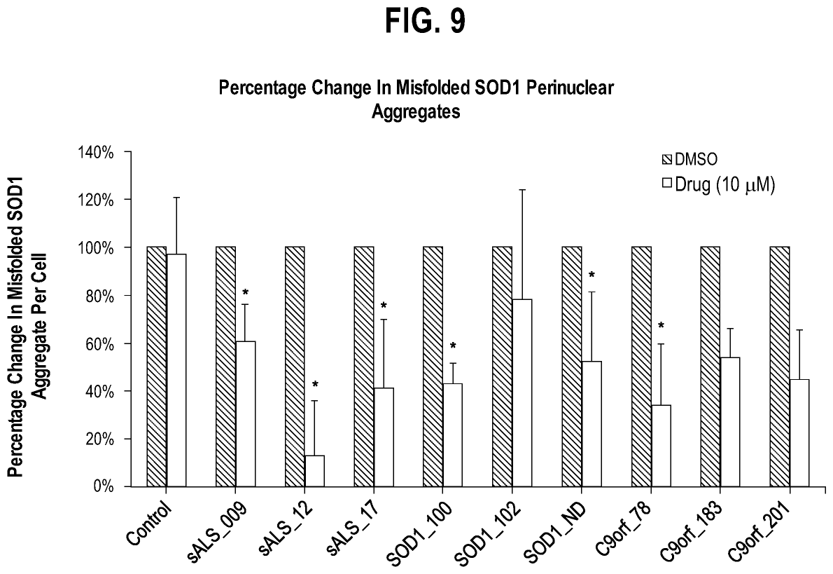

The misfolded SOD1 in iAstrocytes from healthy controls or ALS patients were evaluated with and without andrographolide, (6a5)-6-methyl-5,6,6a,7-tetrahydro-4H-dibenzo[de,g]quinoline-10,11-diol, MMF and riluzole (FIGS. 7, 8 and 9).

Methodology:

The preparation of iAstrocytes has been described in Example 1. At Day 5, the 96 well plate was coated with fibronectin diluted 1:400 in PBS and allowed to set for cell adhesion. iAstrocytes were first washed in an appropriate volume of PBS before incubating for 5 min at 37° C. in lml of accutase. The accutase was neutralized in an appropriate volume of iAstrocyte medium and cells were collected in a 15 ml falcon and centrifuged at 200g for 4 min to form a pellet. The pellet was resuspended in an appropriate volume of medium and the cells were counted using a Burker hemocytometer. The cells were seeded at the desired density and were left for 24 hours to adhere.

Four drugs, i.e., andrographolide, (6aS)-6-methyl-5,6,6a,7-tetrahydro-4H-dibenzo[de,g]quinoline-10,11-diol, MMF and riluzole were made up to a 10 mM stock concentration and diluted 1:1000 in iAstrocytes medium to have a 10 μM working concentration. At day 6, the cells were treated with drugs 24 hours prior to cell assay.

At Day 7, iAstrocytes were fixed in 4% PFA. These were then stained with misfolded SOD1 antibody (B8H10), CD44 to identify cell area and DAPI. Columbus analysis software was used to quantify immunocytochemistry images. In each condition, the number of nuclei was established. In astrocytes stained for misfolded SOD1 protein aggregates, the number, intensity and area of misfolded SOD1 aggregates within the nucleus and surrounding perinuclear area were quantified as well as the percentage of cells positive for misfolded SOD1 accumulation.

### Result:

Columbus analysis software (PE) was able to detect misfolded SOD1 aggregates within the cytoplasm and the perinuclear area of the iAstrocytes, where aggregates are more likely to be identified. Among all parameters, the astrocytes from ALS patients carrying SOD1 mutations had the highest number perinuclear aggregates and percentage of positive cells. Sporadic and C9orf72 lines displayed higher levels than controls. This antibody is specific for misfolded SOD1, with no discrimination between wildtype SOD1 (wtSOD1) and mutant SOD1, and therefore wtSOD1 protein aggregation in control cells can be detected. Treatment of (6aS)-6-methyl-5,6,6a,7-tetrahydro-4H-dibenzo[de,g]quinoline-10,11-diol led to the reduction of misfolded SOD1 positive cells across all cell types, showing the greatest decrease in SOD1 astrocytes. This reduction of misfolded SOD1 in the (6a5)-6-methyl-5,6,6a,7-tetrahydro-4H-dibenzo[de,g]quinoline-10,11-diol treated condition is not seen in the other drug treatments, implying that (6a5)-6-methyl-5,6,6a,7-tetrahydro-4H-dibenzo[de,g]quinoline-10,11-diol may specifically target misfolded SOD1.

1.-50. (canceled)

51. A method of reducing protein m sfolding in a cell or reducing accumulation of misfolded protein in a cell, comprising the step of contacting the cell with a therapeutically effective amount of (6aS)-6-methyl-5,6,6a,7-tetrahydro-4H-dibenzo[de,g]quinoline-10,11-diol,

52. The method of claim 51, wherein the misfolded protein is Cu/Zn superoxide dismutase (SOD1).

53. The method of claim 51, wherein the cell is a cell type or from a tissue selected from any one or more of adrenal gland, bone marrow, brain, breast, bronchus, caudate, cerebellum, cerebral cortex, cervix, uterine, colon, endometrium, epididymis, esophagus, fallopian tube, gallbladder, heart muscle, hippocampus, kidney, liver, lung, lymph node, nasopharynx, oral mucosa, ovary, pancreas, parathyroid gland, placenta, prostate, rectum, salivary gland, seminal vesicle, skeletal muscle, skin, small intestine (including duodenum, jejunum and ileum), smooth muscle, spleen, stomach, testis thyroid gland, tonsil, urinary bladder, or vagina.

54. The method of claim 53, wherein the brain cell is from a brain tissue selected from cerebrum, cerebellum, diencephalon, or brain-stem.

55. The method of claim 54, wherein the brain cell is a neuron, astrocyte, oligodendrocyte, or microglia.

56. The method of claim 55, wherein the neuron is a sensory neuron, motor neuron, interneuron, or brain neuron.

57. The method of claim 51 wherein the cell is a diseased cell.

58. The method of claim 57, wherein the diseased cell is from an animal having a disease or disorder selected from any one or more of aging-related tau astrogliopathy (ARTA), Alexander disease, Alzheimer's disease, amyotrophic lateral sclerosis (ALS), critical illness myopathy (CU), primary age-related tauopathy (PART), aortic medial amyloidosis, ApoAl amyloidosis, ApoAl I amyloidosis, ApoAlV amyloidosis, argyrophillic grain disease, ataxia telangiectasia, atrial fibrillation, autosomal dominant hyper-IgE syndrome, cardiac atrial amyloidosis, Bloom's syndrome, cardiovascular diseases, coronary artery disease, myocardial infarction, stroke, restenosis, arteriosclerosis, cataracts, cerebral amyloid angiopathy, Christianson syndrome, chronic traumatic encephalopathy, Cockayne's syndrome, corneal lactoferrin amyloidosis, corticobasal degeneration, Crohn's disease, Cushing's disease, cutaneous lichen amyloidosis, cystic fibrosis, Dentatorubropallidoluysian atrophy (DRPLA), dialysis amyloidosis, diffuse neurofibrillary tangles with calcification, Down syndrome, endotoxin shock, familial amyloidosis of the Finnish type, familial amyloidotic neuropathy, familial British dementia (FBD), familial Danish dementia (FDD), familial dementia, fibrinogen amyloidosis, fragile X syndrome, fragile X-associated tremor/ataxia syndrome (FXTAS), Friedreich's ataxia, fronto-temporal degeneration, glaucoma, glycogen storage disease type IV (Andersen disease), Guadeloupean Parkinsonism, hereditary lattice corneal dystrophy, Huntington's disease, inclusion body myositisimyopathy, inflammation, inflammatory bowel disease, ischemic condition, ischemia; reperfusion injury, myocardial ischemia, stable angina, unstable angina, stroke, ischemic heart disease and cerebral ischemia, light chain or heavy chain amyloidosis, lysosomal storage diseases, aspartylglucosaminuria, Fabry's disease, Batten disease, Cystinosis, Farber, Fucosidosis, Galactasidosialidosis, Gaucher's disease Type 1, 2 or 3, Gml gangliosidosis, Hunter's disease, Hurler-Scheie's disease, Krabbe's disease, a-mannosidosis, B-mannosidosis, Maroteaux-Lamy's disease, metachromatic leukodystrophy, Morquio A syndrome, Morquio B syndrome, mucolipidosis II, mucolipidosis III, Neimann-Pick disease type A, B or C, Pompe's disease, Sandhoff disease, Sanfilippo syndrome type A, B, C or D, Schindler disease, Schindler-Kanzaki disease, Sialidosis, Sly syndrome, Tay-Sach's disease, Wolman disease, lysozyme amyloidosis, Mallory bodies, medullary thyroid carcinoma, mitochondrial myopathies, multiple sclerosis, multiple system atrophy, myotonic dystrophy, myotonic dystrophy, neurodegeneration with brain iron accumulation, neurofibromatosis, neuronal ceroid lipofuscinosis, odontogenic (Pinborg) tumor amyloid, Parkinsonism-Dementia of Guam, Parkinson's disease, peptic ulcers, Pick's disease, pituitary prolactinoma, post encephalitic Parkinsonism, prion diseases (transmissible spongiform encephalopathies), including Creutzfeldt-Jakob disease (CJD), variant Creutzfeldt-Jakob disease, Gerstmann-Straussler-Scheinker Syndrome, fatal familial insomnia, Kuru, progressive supranuclear palsy, pulmonary alveolar proteinosis, retinal ganglion cell degeneration in glaucoma, retinitis pigmentosa with rhodopsin mutations, seminal vesical amyloid, senile systemic amyloidoses, serpinopathies, sickle cell disease, spinal and bulbar muscular atrophy (SBMA), spinocerebellar ataxias, spinocerebellar ataxia type 1, spinocerebellar ataxia type 2, spinocerebellar ataxia type 3 (Machado-Joseph disease), spinocerebellar ataxia type 6, spinocerebellar ataxia type 7, spinocerebellar ataxia type 8, spinocerebellar ataxia type 17), subacute sclerosing panencephalitis, tauopathies, type II diabetes, vascular dementia, or Werner syndrome.

59. The method of claim 51, wherein the disease or disorder is selected from any one or more of: age-related macular degeneration, Alzheimer's disease (AD), amyotrophic lateral sclerosis (ALS), atherosclerosis, autism spectrum disorder (ASD), benign focal amyotrophy, cerebral infarction, Creutzfeldt-Jakob disease, Crohn's disease, Duchenne's paralysis, Friedreich's ataxia, frontotemporal dementia (FTD), glaucoma, hereditary spastic paraplegia (HSP), Huntington's disease (HD), inclusion body myopathy (IBM), inflammatory bowel disease, ischemia, Kugelberg-Welander syndrome, Lewy body diseases (LBD), Lou Gehrig's disease, multiple sclerosis (MS), myocardial infarction, necrotizing enterocolitis, neurofibromatosis type I, Paget's disease of the bone (PDB), Parkinson disease (PD), primary lateral sclerosis (PLS), progressive bulbar palsy (PBP), progressive muscular atrophy (PMA), pseudobulbar palsy, spinal muscular atrophy (SMA), ulcerative colitis, valosin-containing protein (VCP)-related disorders, or Werdnig-Hoffmann disease_{;} transient ischemic attack, ischaemia, cerebral hemorrhage, senile cataract, retinal ischemia, retinal vasculitis, Brown-Vialetto-Van Laere syndrome, Eales disease, meningitis and encephalitis, post-traumatic stress disorder, Charcot-Marie-Tooth Disease, macular degeneration, X-linked bulbo-spinal atrophy, presenile dementia, depressive disorder, temporal lobe epilepsy, hereditary Leber optic atrophy, cerebrovascular accident, subarachnoid hemorrhage, and schizophrenia.

60. The method of claim 51, wherein the therapeutically effective dose is at least 0.12 mg/kg.

61. The method of claim 51, wherein the therapeutically effective dose is between 5 mg/day and 5000 mg/day.

62. The method of claim 51, wherein the (6aS)-6-methyl-5,6,6a,7-tetrahydro-4H-dibenzo[de,g]quinoline-10,11-diol is administered by oral administration.

63. A pharmaceutical composition comprising (6aS)-6-methyl-5,6,6a,7-tetrahydro-4H-dibenzo[de,g]quinoline-10,11-diol and at least one pharmaceutically acceptable excipient.

64. The pharmaceutical composition of claim 63, wherein the pharmaceutical composition is formulated for oral administration.

65. The pharmaceutical composition of claim 63, wherein the pharmaceutical composition is formulated for subcutaneous administration.
